(19) United States
(12) Patent Application Publication
(10) Pub. No.: US 2015/0094633 A1
(43) Pub. Date: Apr. 2, 2015

(54) POSTURE BRACE WITH BODY ALIGNMENT CAPACITY

(71) Applicant: Jared Paul Garcia, San Francisco, CA (US)

(72) Inventor: Jared Paul Garcia, San Francisco, CA (US)

(21) Appl. No.: 14/499,043

(22) Filed: Sep. 26, 2014

Related U.S. Application Data
(60) Provisional application No. 61/885,137, filed on Oct. 1, 2013.

(51) Int. Cl. A61F 5/02

(52) U.S. Cl. CPC A61F 5/026
USPC 602/19

(57) ABSTRACT

Provided herein is a posture brace with back alignment capacity for improving the posture of a user. The posture brace comprises: an alignment holder for an alignment element and a base element. The alignment holder is attached to the base element, and the alignment holder is configured to position the alignment element for body alignment when the user wears the posture brace. Also provided are system and methods for using the same.

### CROSS-REFERENCE TO RELATED APPLICATION

This application claims priority to U.S. provisional patent application No. 61/885,137, filed Oct. 1, 2013 and entitled “Adjustable Posture Brace,” which is hereby incorporated by reference herein in its entirety.

### FIELD OF THE INVENTION

The present invention relates to the technical field of Health and Fitness. More particularly, the present invention relates to apparatus that can be used in Fitness and Rehabilitation. More particularly, the present invention relates to improved wearable corrective fitness braces with body alignment capacity.

### BACKGROUND

Correct posture is an overlooked necessity of daily life. In modern society, people spend more and more time sitting in front of a TV or a computer. Less time is spent exercising. Fewer people are aware of correct movement patterns and posture. Consequently, proper body functionality has taken a back seat to work pressure, leisure and ignorance. Correct posture allows for the free expression of movement. However, poor postures lead to breathing dysfunction, muscle aches, headaches, muscular pain, sciatica, circulation problems, balance issues, premature spinal arthritis, and many others dysfunctions and health issues.

What is needed are apparatuses for enhance posture awareness and improving posture and body alignment.

### SUMMARY OF THE INVENTION

Provided herein is a posture brace with back alignment capacity for improving the posture of a user. The posture brace comprises: an alignment holder for an alignment element and a base element. The alignment holder is attached to the base element, and the alignment holder is configured to position the alignment element for body alignment when the user wears the posture brace.

In some embodiments, the alignment holder comprises a tubular pouch having a first end, a second end, and a cavity from the first end to the second end.

In some embodiments, at least one of the first and second ends of the pouch can be closed and opened via a closure mechanism. In some embodiments, the closure mechanism is selected from the group consisting of a Velcro closure mechanism, a button closure mechanism, a drawstring closure mechanism, an elastic closure mechanism, and a combination thereof.

In some embodiments, the one of the first and second ends is closed. In some embodiments, the alignment holder comprises a plurality of holder elements. Each holder element in the plurality has a orifice through which an alignment element can be positioned and the orifices corresponding to the plurality of holder elements are aligned in a pre-determined fashion. In some embodiments, the pre-determined fashion is approximately a straight line.

In some embodiments, the base element comprises a back support element or a shoulder support element. In some embodiments, the base element is selected from the group consisting of a vest; two shoulder straps connected to a back support element; and two shoulder straps and a belt each connected to the alignment holder. In some embodiments, the body alignment is selected from the group consisting of back alignment, torso alignment, neck alignment, shoulder alignment, leg alignment, and a combination thereof.

In another aspect, provided herein is a posture alignment system that comprising the posture brace as disclosed herein and an alignment element. The alignment element is positioned in the alignment holder of the posture brace to provide body alignment.

In some embodiments, the alignment element is made of a material selected from the group consisting of plastic, wood, metal, a synthetic polymeric material, polystyrene, polycarbonate, polyvinyl chloride, polyethylene, polypropylene, and a combination thereof. In some embodiments, the alignment element is extendible.

In some embodiments, in non-extended state, the alignment element is entirely received within the alignment holder.

In some embodiments, the alignment element is entirely received within the alignment holder.

In some embodiments, the alignment element is round, square, rectangle, or contoured to fit the back of a user wearing the posture brace.

In another aspect, provided herein are methods of exercising or performing still or slow motions while using a posture brace or posture brace system as disclosed herein.

It will be understood that any embodiment disclosed herein can be used in any aspect, alone or in combination with another embodiment or other embodiments.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates an exemplary embodiment, attached to adjustable body braces with adjustable shoulder straps and belt buckle.

FIG. 2 illustrates an exemplary embodiment, attached to fit vest.

FIG. 3 illustrates an exemplary embodiment, in which a body alignment rod is positioned along the spine of a user.

FIG. 4A illustrates an exemplary embodiment of a pouch-like holder for positioning a body alignment rod.

FIG. 4B illustrates an exemplary embodiment of a pouch-like holder for positioning a body alignment rod.

FIG. 4C illustrates an exemplary embodiment of a pouch-like holder for positioning a body alignment rod.

FIG. 5A illustrates an exemplary embodiment of two separate holders for positioning a body alignment rod.

FIG. 5B illustrates an exemplary embodiment of two separate holders for positioning a body alignment rod.

FIG. 5C illustrates an exemplary embodiment of two separate holders for positioning a body alignment rod.

FIG. 6 illustrates an exemplary embodiment of an extendible body alignment rod.

FIG. 7A illustrates an exemplary embodiment used for alignment during slow motion.

FIG. 7B illustrates an exemplary embodiment used for static alignment work.

FIG. 8A illustrates an exemplary embodiment used for alignment during workout.

FIG. 8B illustrates an exemplary embodiment used for alignment during workout.

FIG. 9A illustrates an exemplary embodiment used for alignment during workout with length adaption.

FIG. 9A illustrates an exemplary embodiment used for static alignment with length adaption.

FIG. 10A illustrates an exemplary embodiment used in combination with other exercise equipment.

FIG. 10B illustrates an exemplary embodiment used in combination with other exercise equipment.

### DETAILED DESCRIPTION OF THE INVENTION

Provided herein are apparatuses and methods for using the same to improve physical postures of a user. The apparatus aim to solve a few posture-related issues by focusing on both static and dynamic proper functions of human body, e.g., the alignment of torso, neck, shoulders, legs and other parts.

The apparatuses are interchangeably referred to as “wearable corrective posture brace,” “body alignment brace,” “body alignment system,” or “alignment system.”

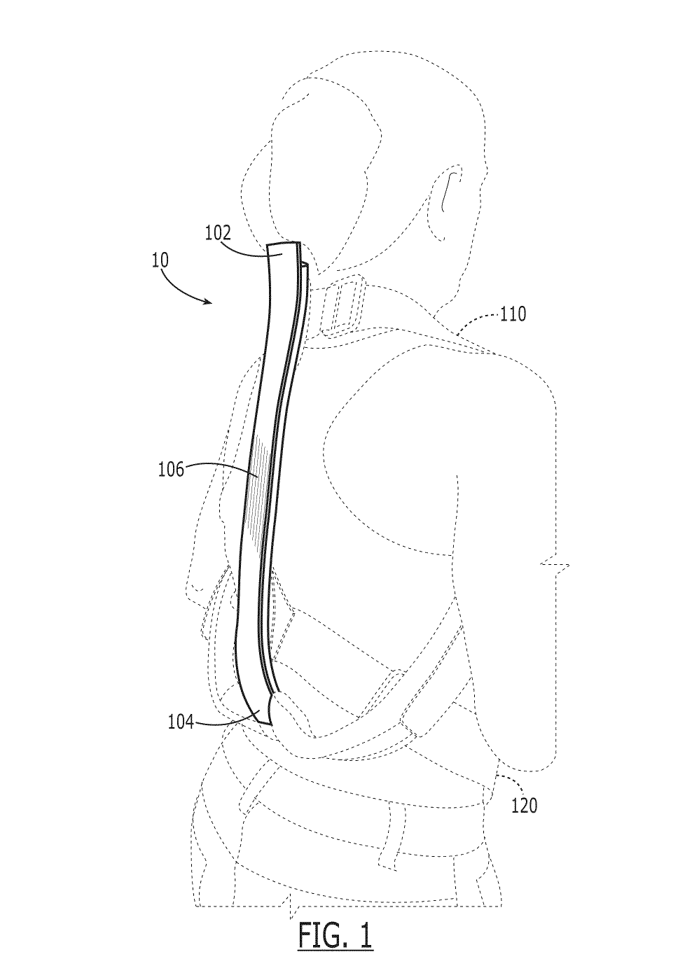

Referring to FIG. 1, an exemplary embodiment of a posture brace 10 include a holder element 106 and a base element to which the holder is attached. In some embodiment, base element comprises adjustable shoulder straps 110 and a belt 120. In some embodiments, holder element 106 is a long tubular structure having two ends: a first end 102 and a second end 104. In some embodiments, one of the two ends, e.g., second end 104, is open. In some embodiments, one of the two ends, e.g., second end 104, is closed. When a user wears posture brace 10, the long dimension of tubular pouch-like holder 106 is aligned or proximately aligned with the spine with the user.

Both shoulder straps and belts have adjustable mechanisms that allow a user to adjust and refine the fit of the shoulder straps and belt as well as the alignment of the holder element (consequently the alignment rod that is placed with the holder element). For example, such mechanisms also allow a user to position and fine tune the alignment of the holder element such that top end 102 and bottom end 104 fall on a line that goes over the spine of the particle user. In some embodiments, a user can adjust the position and alignment of holder element 106 using shoulder straps 110. In some embodiments, a user can adjust the position and alignment of holder element 106 using belt 120. In some embodiments, both shoulder straps and belts can be adjusted to ensure proper alignment and position. In some embodiments, different positions on a shoulder strap are labeled according to the resulting sizes when a user adjusts the shoulder element to the corresponding positions. In some embodiments, the shoulder straps are labeled as extra-small, small, medium, large or extra-large or in similar fashions to denote different sizes.

Body alignment as described herein is not limited to spine or torso alignment. In some embodiments, body alignment in accordance with the present invention includes the alignment of the torso with the lower body. In some embodiments, body alignment in accordance with the present invention includes the alignment of the head with the torso. In some embodiments, body alignment in accordance with the present invention includes the alignment of the head with the torso. In some embodiments, body alignment in accordance with the present invention includes the horizontal alignment of shoulders. Spine or torso alignment is described herein as an example and should not be held as a limitation to the scope of the invention.

Any suitable shoulder straps or belts (for example, those used in backpacking gears) can be used in these embodiments. Exemplary should straps and belts include but are not limited to those described in U.S. Pat. No. 8,267,290; U.S. Pat. No. 6,141,835; U.S. Pat. No. 6,308,381; U.S. Pat. No. 3,063,118; US Pat. Pub. No. 2012/0126607; US Pat. Pub. No. 2005/0255791; each of which is hereby incorporated by reference herein in its entirety.

Any known posture braces can function as a base element in accordance with the present invention. Exemplary base elements include but are not limited to those disclosed in U.S. Pat. No. 3,116,735; U.S. Pat. No. 3,292,616; U.S. Pat. No. 4,751,923; U.S. Pat. No. 8,047,893; U.S. Pat. No. 4,080,962; US Pat. Pub. No. 2012/0078149; US Pat. No. D499806; US Pat. Pub. No. 2012/0289874; US Pat. Pub. No. 2013/0296756; US Pat. Pub. No. 2012/0278966; and U.S. Pat. No. 8,556,840 each of which is hereby incorporated by reference herein in its entirety.

Further, it is possible to modify any existing piece of clothing to add the body alignment function. For example, a holder element 106 can have one or more pieces of magnets embedded in small pockets. Magnets of opposite polarity can be used to attached holder element 106 to the back of any piece of clothing to add back alignment functionality. Here, the magnets function as the base element that allows the holder element to be positioned along the spine of a user. In preferred embodiments, the magnets have a flat dimension so that they do not create discomfort.

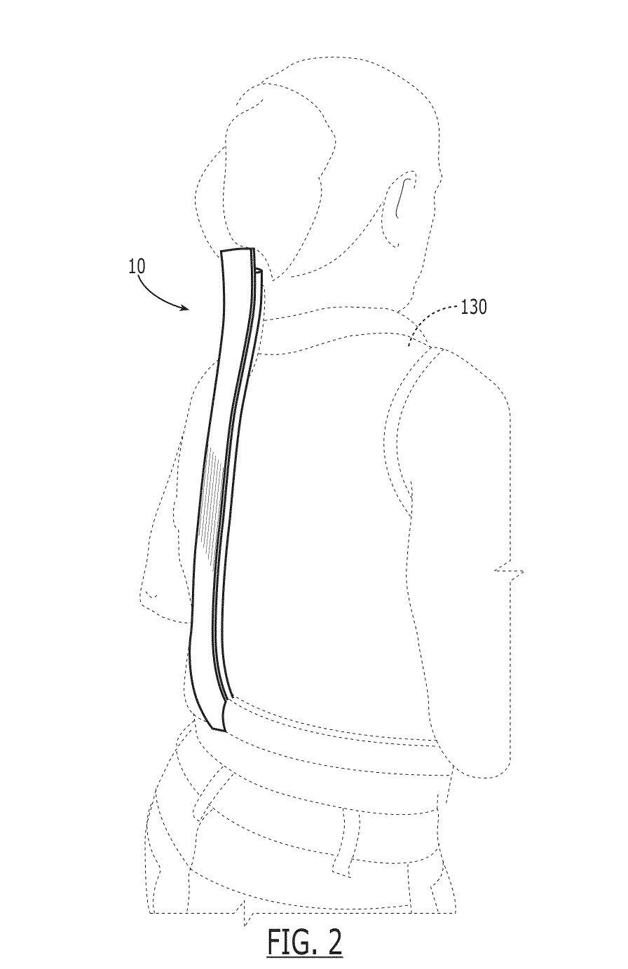

FIG. 2 shows another exemplary embodiment where holder element 106 is part of a piece of clothing; e.g., a fitted vest 130 functions as the base element to which. In some embodiments, the posture brace is permanently attached to the piece of clothing. In some embodiments, the posture brace is attached to the piece of clothing via a removable mechanism; for example, a Velcro™ fastening mechanism, a button mechanism, a zipper mechanism, and etc. In embodiments where the posture brace is permanently attached, the longest dimension of the tubular pouch-like holder is already aligned with the center line along the back of the piece of clothing (and subsequently the spine of a user who wears the piece of clothing. In removable attached posture brace embodiments, a user can align and adjust the tubular pouch-like holder prior to wearing the piece of clothing to ensure proper alignment of the tubular pouch-like holder.

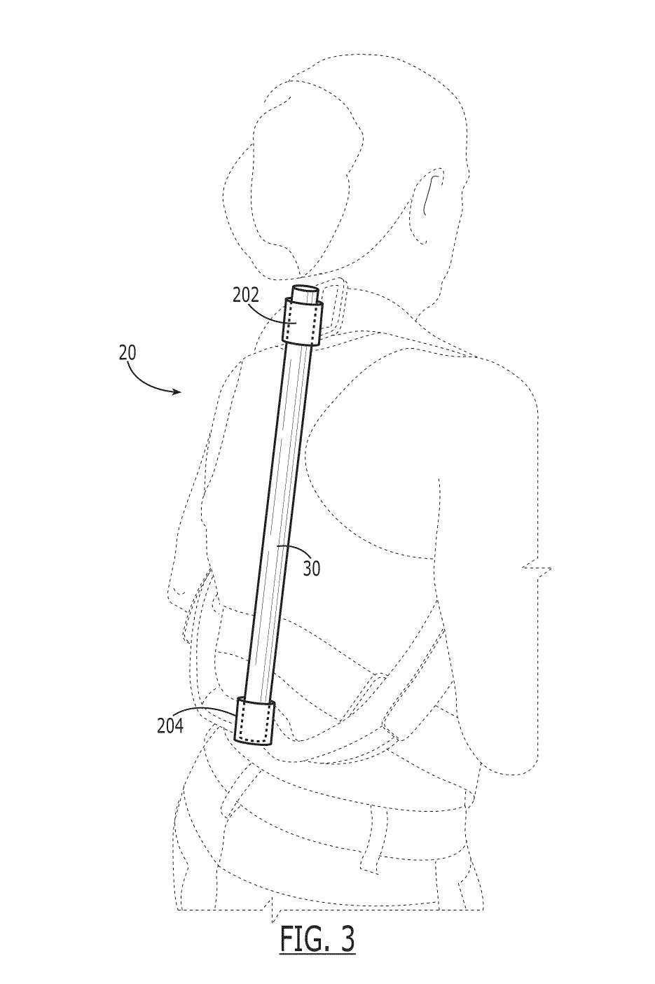

FIG. 3 illustrates an exemplary embodiment in which a back alignment rod 30 is fitted through holder 106. In this embodiment, holder 106 comprises two tubes that are aligned in a straight line (e.g., elements 202 and 204 in FIG. 3). In some embodiments, top element 202 is open at both ends while the bottom tube is closed at the bottom to hold the alignment rod. In some embodiments, one or more additional tubes can be used.

In some embodiments, all or a portion of the holder element is elastic to securely hold alignment rod 30. In some embodiments, all or a portion of the holder element has a fastening mechanism to securely hold alignment rod 30. Such a fastening mechanism includes but is not limited to a draw-string, a button, a Velcro™, and etc.

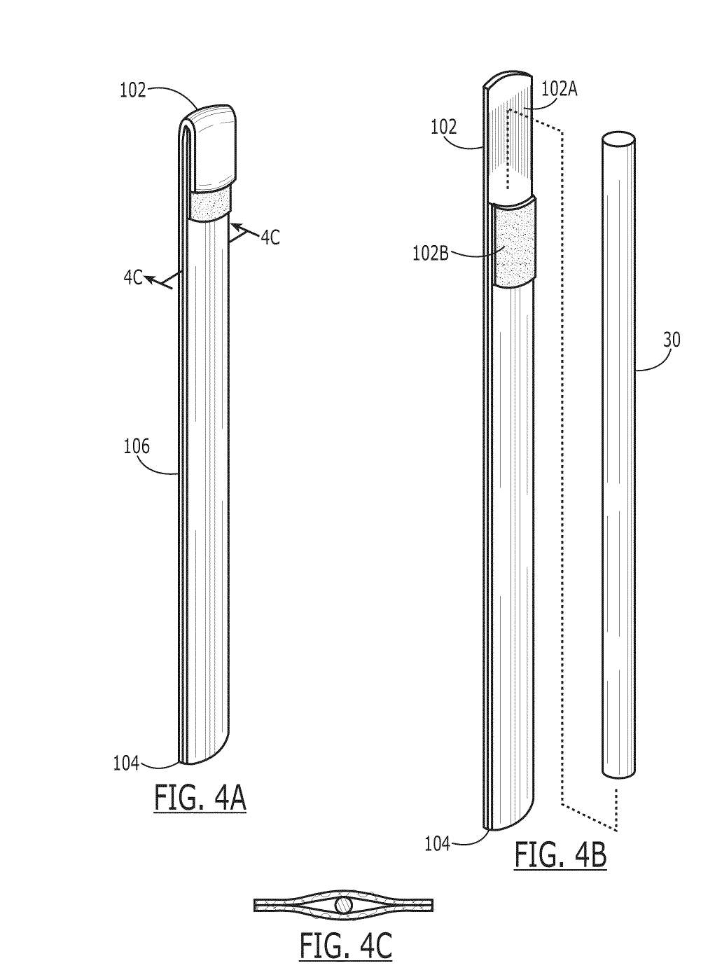

FIGS. 4A through 4C illustrate an exemplary embodiment of the holder element 206. The holder 106 is a tubular pouch, having a top end 102 and a bottom end 104 and a thin linear body. In this example, bottom end 104 is closed. Top end 102 has a Velcro™ based closure mechanism comprising element 102A and element 102B. Element 102A and element 102B can be reversibly closed (e.g., FIG. 4A) or opened (e.g., FIG. 4B). Holder 106 has a cavity along the thin linear body, in which alignment rod 30 can be positioned, as illustrated in FIGS. 4B and 4C.

In some embodiments, size of a holder element (e.g., the length of the thin linear body) can be adjusted according to the height and spine length of a user. In some embodiments, a pouch-like holder (e.g., shown in FIGS. 1, 2, and 4) is made of flexible fabrics such that the pouch can be stretched to adopt to the size of a particular user. In some embodiments, the pouch like holder has many folded features along the length, which can be extended adopt to the size of a particular user. In other embodiments where two or more tube-like elements are used to position an alignment rod (e.g., FIG. 3), the relative distance of the tube-like elements can be adjusted so that they can best hold an alignment rod that is most suitable for a particular user. For example, there can be multiple Velcro™ patches along the back center of a fitted vest. A user can adjust the distance between two tube-like elements by attaching them to different patches. In some embodiment, the bottom tube-like element of a holder element is permanently attached to either adjustable shoulder straps, a belt or a piece of clothing, while the position of top tube-like element is adjusted according to the size (e.g., the height or torso length of a user). In such embodiments, positions for the top tube-like element can be labeled, for example, as extra-small, small, medium, large and extra-large or in similar fashions to denote different sizes. In some embodiments, the top tube-like element is fixed while the position of the bottom tube-like element is adjustable. In some embodiments, positions of both top tube-like element and bottom tube-like element are adjustable.

The holder element can be made of any applicable material, including for example but not limited to, synthetic or national fabrics. In some embodiments, padding can be added to provide comfort. In some embodiments, a thin layer of material forms the holder while fit and comfort is provided by the alignment element such as an alignment rod. In some embodiment, the holder element has pores for transmit perspiration.

In some embodiments, auxiliary features can be added to the holder element to provide additional functionalities. For example, light-weight plastic or rings, ring-like or other structures can be fixed along the sides of a holder element. In some embodiments, a user can clip weights (such as sand bags) onto the ring or other structures to add weight training while a user is attempting posture correction.

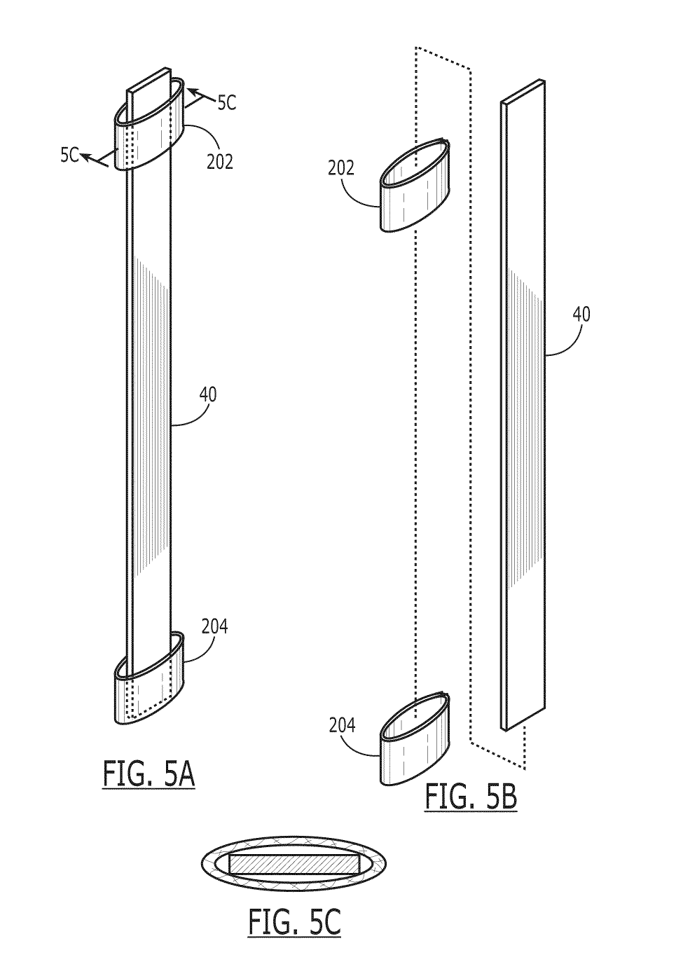

In some embodiments, an improved wearable corrective posture brace is an alignment system that further comprises an alignment element, such as a straight alignment rod. Despite its name, an alignment rod (e.g., element 30 in FIG. 4B) is not limited to a particular size or shape. For example, an alignment rod 30 from FIG. 4B has a circular cross-section. Alignment rod 40, as depicted in FIGS. 5A through 5C, adopts a ruler-like conformation with a rectangular shaped cross-section. There is no limitation on the shape of an alignment rod so long as its longest dimension can be aligned with a part of the body of a user. Applicable shapes of a cross-section of an alignment rod include but are not limited to a circle, an oval, a square, a rectangle, a triangle, or an irregular shape which partially conforms to the contour of the part of the body when it is properly aligned.

In some embodiments, when the alignment system is used for children, the alignment rod can be bright colored and have shapes that are of interest to children, for example, flowers, stars and etc.

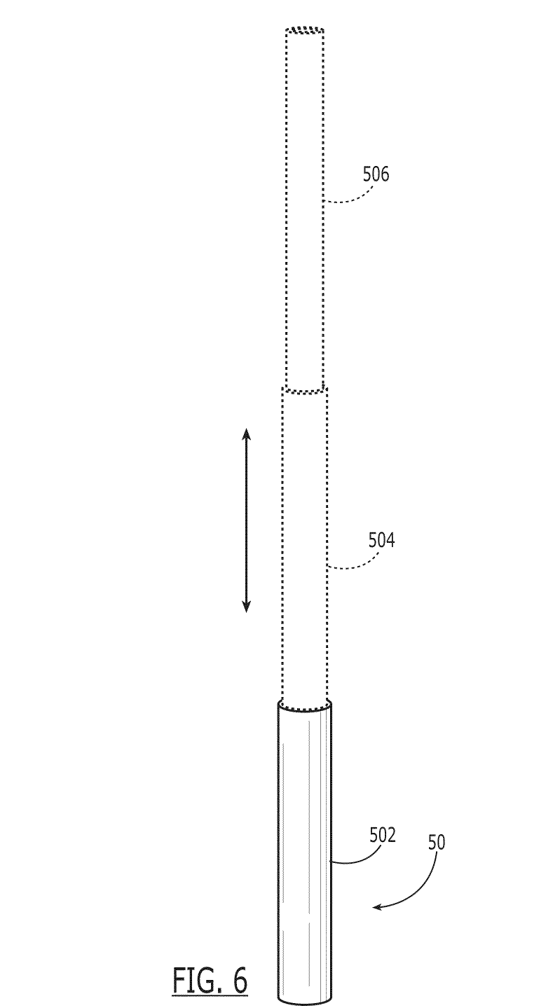

An alignment rod can have different lengths, width, or circumference. In some embodiments, the length, width or circumference of an alignment rod is adjustable. FIG. 6 depicts an extendible alignment rod. The configuration of an extendible alignment rod is similar to that of an extendible TV antenna. For example, alignment rod 50 comprises elements 502, 504 and 506, which have gradually smaller diameters. The element 502, having with the smallest diameter, is sheathed in element 504, which has an internal diameter match the outer diameter of element 504. Element 504 is in turn sheathed within element 506. Although elements 502, 504 and 506 are depicted as having a circular shape, one of skill in the art would understand that these elements can adopt any shape so long as the outer-dimension of the smallest one fits the inter-dimension of the element at the next size up, in a manner similar to that of a set of Russian dolls.

In a non-extended or collapsed state, element 506 is pushed into element 504, which in turn is pushed into element 502. In an extended state, e.g., as shown in FIG. 6, element 506 is pulled out of or extended from element 504, which is in turn pulled out of or extended from element 50. The length of the alignment rod can be adjusted by the number of elements that are in extended state versus the number of elements that are in non-extended or collapsed state. In a collapsed state, the entire alignment rod can be fit into the holder element (as shown in FIG. 4A).

Any suitable material can be used to form the alignment element, including but not limited to plastic, wood, metal, synthetic polymeric material such as polystyrene, polycarbonate, polyvinyl chloride (PVC), polyethylene, Acrylonitrile butadiene styrene (ABS), hard vinyl, polyester nylon, and polypropylene. In some embodiments, a lightweight PVC pipe or pole is used as the alignment element. In some embodiments, a heavy weight material is used to construct the alignment element to incorporate weight training into alignment training

In some embodiments, an alignment element (e.g., element 40 in FIGS. 5A and 5B) is embedded in a concealed back pocket of a piece of clothing (e.g., a sports vest). In some embodiments, the concealed alignment has a predetermined length to match the size of the piece of clothing and in turn the person wearing the piece of clothing. In some embodiment, the length of the alignment element can be adjusted by adopting the Russian doll configuration.

Depending on the purposes, a user can select an alignment rod according to, for example, its circumference, shapes and material type. For example, for only torso alignment, a short alignment rod of light weight can be used. For whole body alignment, a long alignment rod can be used. In some embodiments, a metal alignment rod can be used. In some embodiments, multiple weights (e.g., sand bags and small metal weights) can be attached to add strength training to body alignment.

In some embodiments, a posture brace of the present invention is used for static alignment when the user is sitting or doing slow motion. In some embodiments, a posture brace of the present invention is used for functional alignment during the user does continuous movement.

In some embodiments, a posture brace of the present invention is used in conjunction with other exercise equipment when applicable, including but not limits to, for example, dumbbells, weight-lifting equipment, stationary bikes, Free weights, cardio equipment, Vipr™, TRX™, BOSU™, Kettlebells™, medicine balls, weight machines, resistance training machines, and etc.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Exemplar Sizes

Piece along the spine: height—8-27 inches, width 2-3 inches; alignment holder: height—5-25.75 inches, width 2.25 inches; Velcro: width—1 -2 inches; attachment site for arm straps: length about 5.75 inches, width is about 5 inches.

Arm Straps: length—25 to 53 inches, width about 2 -3 inches; softer portion (goes around user's shoulders): length 15-38.5 inches, width—3 inches; Nylon portion: length—24 inches, width—2 inches; Nylon Velcro: length—5.75 inches to 12 inches from the end of the arm strap, width—1 inches; softer portion Velcro: length—17 inches, width—1 inches

Belt: length—25 to 76 inches, width about 2 inches ; belt buckle: length—2.5 inches, width—2.5 inches.

### Example 2

### Alignment During Slow Motion or When in Static Position

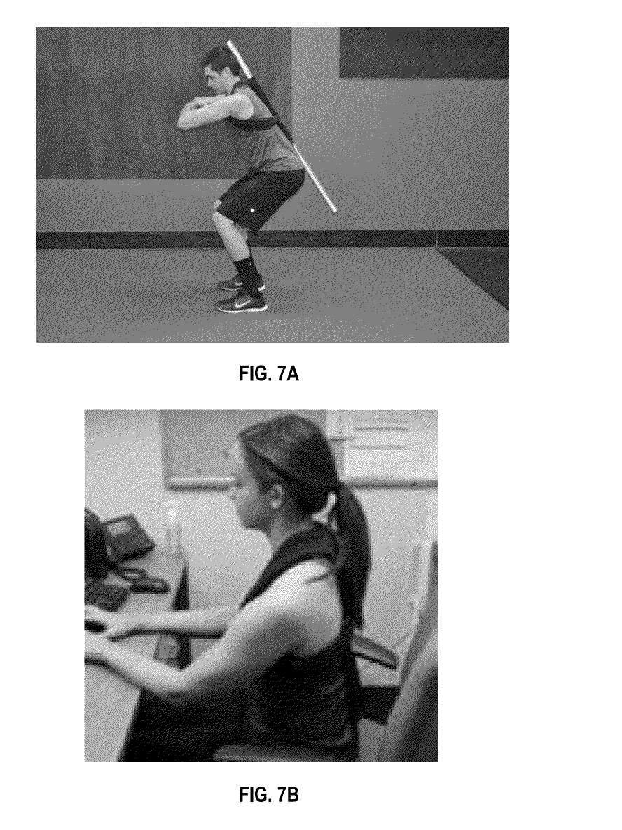

Referring to FIG. 7A, a user wearing a belt-less posture brace with a long alignment element is doing slow motion body alignment work. FIG. 7B shows a user wearing a belt-less posture brace with an embedded alignment element while performing desk working.

### Example 3

### Alignment During Exercise

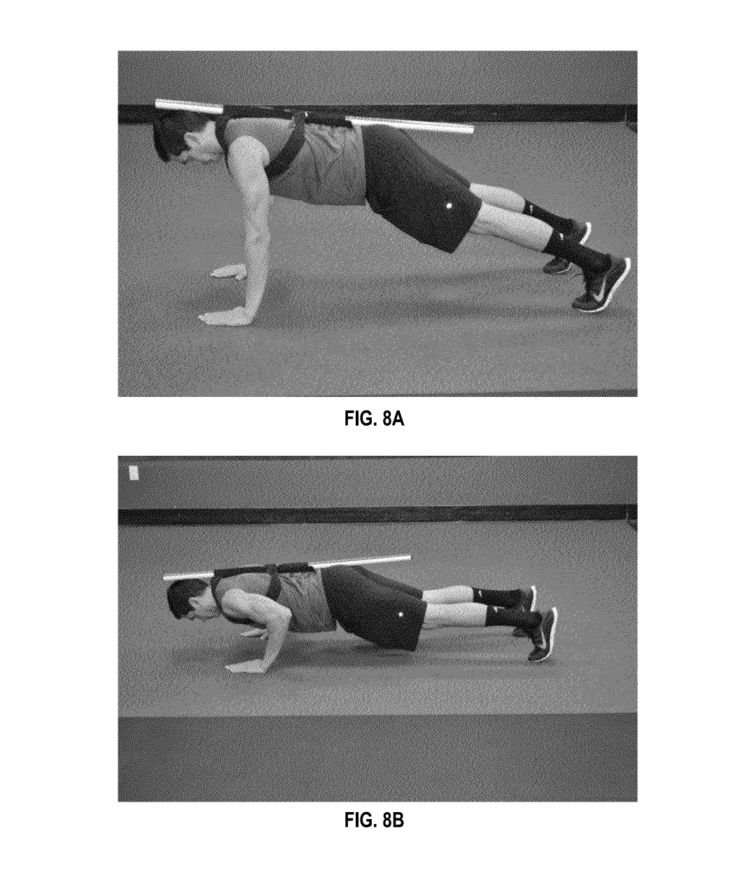

FIGS. 8A and 8B show a user wearing a belt-less posture brace with a long alignment element while doing consecutive movements. This way, the user has a better sense of body alignment while exercising, for example, while doing push-ups.

### Example 4

### Optional Alignment

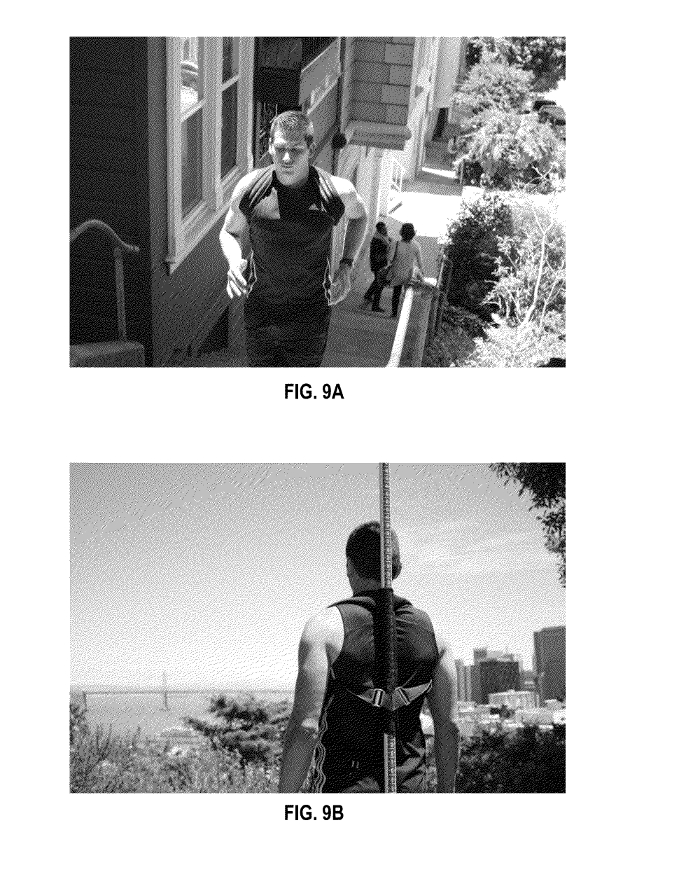

FIG. 9A depicts a user running wearing a posture brace. The user has the option to choose a very short alignment element so the user can maintain upper body posture without interference during exercise. The user could even choose to take out the alignment element during the run. When the user stands still for breathing exercise (FIG. 9B), he uses an extended alignment element to help body alignment.

### Example 5

### Application in Conjunction with Other Equipments

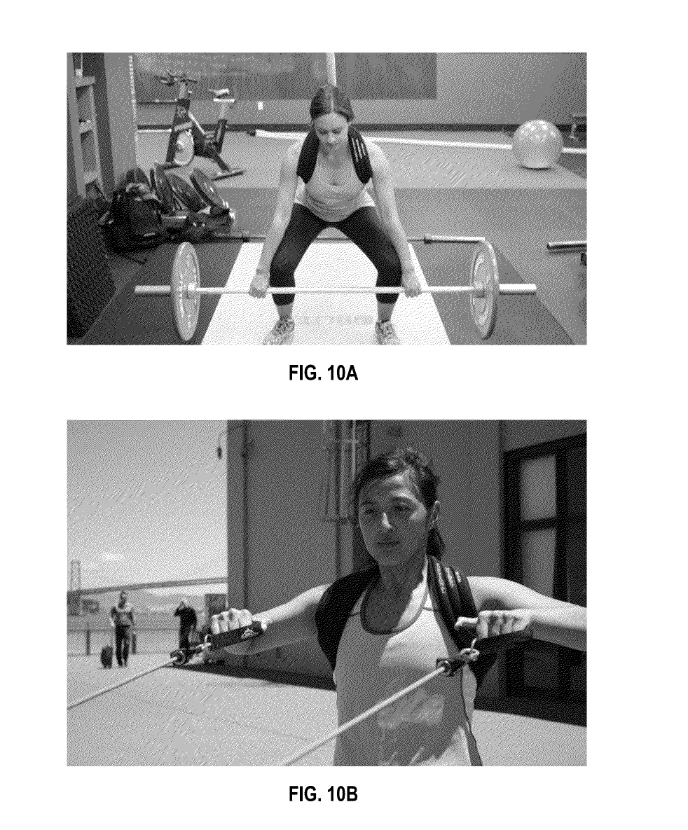

FIGS. 10A and 10B show that the posture brace with or without a belt, can be used while the user is using that exercise equipment. For example, users can choose to use a posture brace for upper body alignment while doing weight-lifting (FIG. 10A) or doing pulling exercise (FIG. 10B).

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

We claim:

1. A posture brace with back alignment capacity for improving the posture of a user, comprising:
an alignment holder for an alignment element; and
a base element;
wherein the alignment holder is attached to the base element, and wherein the alignment holder is configured to position the alignment element for body alignment when the user wears the posture brace.

2. The posture brace of claim 1, wherein the alignment holder comprises a tubular pouch having a first end, a second end, and a cavity from the first end to the second end.

3. The posture brace of claim 2, wherein at least one of the first and second ends of the pouch can be closed and opened via a closure mechanism.

4. The posture brace of claim 3, wherein the closure least is selected from the group consisting of a Velcro closure mechanism, a button closure mechanism, a drawstring closure mechanism, an elastic closure mechanism, and a combination thereof.

5. The posture brace of claim 2, wherein one of the first and second ends is closed.

6. The posture brace of claim 1, wherein the alignment holder comprises a plurality of holder elements, wherein each holder element in the plurality has a orifice through which an alignment element can be positioned and the orifices corresponding to the plurality of holder elements are aligned in a pre-determined fashion.

7. The posture brace of claim 6, wherein the pre-determined fashion is approximately a straight line.

8. The posture brace of claim 1, wherein the base element comprises a back support element or a shoulder support element.

9. The posture brace of claim 1, wherein the base element is selected from the group consisting of a vest; two shoulder straps connected to a back support element; and two shoulder straps and a belt each connected to the alignment holder.

10. The posture brace of claim 1, wherein body alignment is selected from the group consisting of back alignment, torso alignment, neck alignment, shoulder alignment, leg alignment, and a combination thereof.

11. A posture alignment system, comprising:
a posture brace of claim 1, and
an alignment element, wherein the alignment element is positioned in the alignment holder of the posture brace to provide body alignment.

12. The posture alignment system of claim 11, wherein the alignment element is made of a material selected from the group consisting of plastic, wood, metal, a synthetic polymeric material, polystyrene, polycarbonate, polyvinyl chloride, polyethylene, polypropylene, and a combination thereof.

13. The posture alignment system of claim 11, wherein the alignment element is extendible.

14. The posture alignment system of claim 13, wherein in non-extended state, the alignment element is entirely received within the alignment holder.

15. The posture alignment system of claim 11, wherein the alignment element is entirely received within the alignment holder.

16. The posture alignment system of claim 11, wherein the alignment element is round, square, rectangle, or contoured to fit the back of a user wearing the posture brace.
